(19) United States
(12) Patent Application Publication
(10) Pub. No.: US 2018/0153383 A1
(43) Pub. Date: Jun. 7, 2018

(54) SURGICAL TISSUE RECOGNITION AND NAVIGATION APARATUS AND METHOD

(71) Applicant: KYPHON SARL, Neuchatel (CH)

(72) Inventors: Mojan Goshayesh, Atherton, CA (US); Travis Nolan, Collierville, TN (US); Michael Smith, San Jose, CA (US)

(73) Assignee: KYPHON SARL

(21) Appl. No.: 15/885,137

(22) Filed: Jan. 31, 2018

Related U.S. Application Data
(62) Division of application No. 14/334,322, filed on Jul. 17, 2014.

(51) Int. Cl. A61B 1/00; G06T 7/40; A61B 90/20; G06T 7/11; A61B 34/20; A61B 1/313

(52) U.S. Cl. CPC A61B 1/00188; G06T 7/40; A61B 90/20; G06T 7/11; A61B 1/00009; A61B 1/0005; A61B 1/00193; A61B 2034/2065; A61B 1/3132; G06T 2207/10012; G06T 2207/10056; G06T 2207/10068; G06T 2207/20092; A61B 34/20

(57) ABSTRACT

A system and method images first and second views of a subject and analyses the view to identify devices and anatomical structures. The images are overlayed with labeling identifying anatomical structures and/or segment regions corresponding to the anatomical structures. The segment regions have indicia differentiating the segment regions. The overlayed images are displayed to a doctor executing a procedure on the subject to assist in identifying the anatomical structures. In an embodiment a nominal anatomical model is adapted to an anatomical structure and substituted in a display to provide greater clarify than an actual image. Devices in the first and second views are identified and optionally movement paths of the devices is tracked and displayed. User input is optionally accepted to adapt the segment regions and/or the adaptation of the anatomical model to the imaged anatomical structure.

The present application is a divisional of U.S. application Ser. No. 14/334,322 filed Jul. 17, 2014; all of which is incorporated herein by reference.

### TECHNICAL FIELD

The present disclosure relates to a system and method for assisting a surgeon executing minimally invasive surgery.

### BACKGROUND

Traditional “open” surgery techniques require opening an incision to create a field of view inclusive of a targeted structure or organ and a surrounding area. Viewing the surrounding area allows a surgeon to recognize various landmarks so as to correctly identify tissues, organs, nerves, blood vessels, and other anatomical items, which provide a frame of reference for the operating procedure. Additionally, a sufficient opening is required for a surgeon to use standard surgical devices. However, traditional “open” surgery has various drawbacks. For example, traditional open lumbar (back) surgeries require a 5- to 6-inch incision which results in damage to the involved tissues. Muscle dissection and retraction for exposing the spine results in formation of scar and fibrotic tissue. The incision requires blood vessel cauterization. Disruption of the anatomy of the spine is thus needed to effect decompression of pinched nerves and installation of screws and devices to stabilize the spine in traditional “open” surgery which can result in lengthy hospital stays, prolonged pain and recovery periods, the need for postoperative narcotic use, significant operative blood loss, and risk of tissue infection.

As an alternative to traditional “open” surgery, minimally invasive surgery (MIS), also called laparoscopic surgery, is conducted in order minimize damage to surrounding tissues and the possibility of infection due to a large incision. In MIS a small incision is made which is large enough for the insertion of devices specifically designed for MIS. As the name implies, laparoscopic surgery requires use of a laparoscope. Laparoscopes may employ a telescopic rod lens system, that is usually connected to a video camera, or, in the case of a digital laparoscope, a charge-coupled device is placed at the end of the laparoscope and electrically coupled to a video display device, eliminating the rod lens system. Also, there is a. fiber optic cable system connected to a ‘cold’ light source to illuminate the operative field. These devices are inserted through a cannula or trocar which has a diameter on the order of 5 mm or 10 mm to view the operative field.

MIS techniques have been developed to treat disorders of the spine with less disruption to the muscles. The camera provides surgeons with a view from inside the cannula, enabling surgical access to the affected area of the spine. Concurrent with use of the camera system, a fluoroscope is also employed to assist the surgeon in determining the position of surgical instruments relative to the spinal column. The fluoroscope provides an x-ray image of the spine wherein the bone structure and surgical device are visible.

While the use of a camera and fluoroscope assist the surgeon in following the path of surgical instruments and viewing small areas of tissue, a problem exists in that operating via the cannula provides an extremely narrow field-of-view (FOV), what may be termed tunnel vision. This makes it difficult to differentiate tissues because a surrounding area is not visible making it impossible to view the overall tissue structures which otherwise assist in recognition of the type of tissue being viewed. The narrow FOV also makes it difficult to determine where a particular portion of tissue or bone is relative to the overall anatomy of the spine. Hence, it is desirable to develop equipment and methods which assist the surgeon in both identifying; tissue types and bone portions, and in knowing where a particular portion is in relation to the overall structure of the spine. In particular, since the narrow FOV of the cannula obstructs view of various landmark anatomical structures, it becomes difficult to differentiate tissue types from each other as location of tissue types relative to landmarks assists in identifying the tissue types, thus a means of tissue type identification is needed.

### SUMMARY

Briefly stated, an embodiment of the present disclosure provides a system and method that images first and second views of a subject and analyses the views to identify devices and anatomical structures. The images are overlayed with labeling identifying anatomical structures and/or segment regions corresponding to the anatomical structures. The segment regions have indicia differentiating the segment regions, The overlayed images are displayed to a doctor executing a procedure on the subject to assist in identifying the anatomical structures. In a further embodiment a nominal anatomical model is adapted to an anatomical structure and substituted in a display to provide greater clarify than an actual image. Devices in the first and second views are identified and optionally movement paths of the devices is tracked and displayed. Yet another embodiment optionally accepts user input to adapt the segment regions and/or to adapt the nominal anatomical model to the imaged anatomical structure.

In certain embodiments of the present disclosure the first view that is imaged is provided by optical means, for example, a stereomicroscope, microscope, or endoscope, while the second view that is imaged is provided by other imaging modalities such as, for example, MRI, flouroscopes, CT, or other devices using techniques other than optical. With regard to the first view using the optical means, the images produced are optionally or preferably processed continuously to provide near-real-time (NRT) views. This can be advantageous to a surgeon in avoiding damage to sensitive tissues such as neural structures in that the surgeon can view incisions in NRT and halt further incising when the sensitive tissues are reached.

In another embodiment of the present disclosure the nominal anatomical model is optionally replaced by a reconstructed 3D anatomical model of the actual patient based on data from preoperative CT or MRI images.

The above, and other objects, features and advantages of the present application will become apparent from the following description read in conjunction with the accompanying drawings, in which like reference numerals designate the same elements. The present application is considered to include all functional combinations of the above described features and is not limited to the particular structural embodiments shown in the figures as examples. The scope and spirit of the present application is considered to include modifications as may be made by those skilled in the art having the benefit of the present disclosure which substitute, for elements presented in the claims, devices or structures upon which the claim language reads or which are equivalent thereto, and which produce substantially the same results associated with those corresponding examples identified in this disclosure for purposes of the operation of this application. Furthermore, operations in accordance with methods of the description and claims are not intended to be required in any particular order unless necessitated by prerequisites included in the operations. Additionally, the scope and spirit of the present application is intended to be defined by the scope of the claim language itself and equivalents thereto without incorporation of structural or functional limitations discussed in the specification which are not referred to in the claim language itself. Accordingly, the detailed description is intended as illustrative in nature and not limiting the scope and spirit of the present application.

### BRIEF DESCRIPTION OF THE DRAWINGS

The present disclosure will become more readily apparent from the specific description accompanied by the following drawings, in which:

FIG. 1 is a block diagram of a navigation system according to an embodiment of the present application;

FIG. 2a is an illustration of an open spinal surgery procedure;

FIG. 2b is an illustration of a minimally invasive surgery (MIS) procedure;

FIG. 2c is a schematic depiction of spinal and surround tissue anatomy indicating a course of surgical procedures;

FIG. 3 is a picture of tissues exposed during an MIS procedure;

FIG. 4 is a flow chart of an embodiment of a microscope image processing procedure an operating procedure according to an embodiment of the present disclosure;

FIG. 5 is a block diagram of an image processing embodiment of the present disclosure;

FIG. 6 is a picture of a rendering produced by the image processing embodiment illustrating segmentation and tissue identification;

FIG. 7 is a flow chart of an embodiment an image processing procedure for processing image data from an imaging device;

FIG. 8 is an illustration of a display showing images produced by image processing according to an embodiment of the present disclosure;

FIG. 9 is another illustration of a display showing images produced by image processing according to another embodiment of the present disclosure;

FIG. 10 is a drawing of front and side elevation views of a model vertebra according to an embodiment of the present disclosure;

FIG. 11 is an alternative block diagram of the navigation unit 38 shown in FIG. 1; and

FIG. 12 is another illustration of displays showing images produced by image processing according to a further embodiment of the present disclosure wherein operation of a zoom feature supplements a first view with details of a second view outside a field-of-view of the first view.

### DETAILED DESCRIPTION

In some embodiments the present disclosure provides a navigation system for displaying devices relative to a subject and identifying anatomical parts of the subject during a surgical procedure executed by a user upon the subject. A first imaging device provides a view of the subject and produces first image data representative of the view. The first imaging device is configured to receive second image data, generate an overlay image from the second image data, and superimpose the overlay image on the view of the subject. An image segmentation unit receives the first image data, effects image processing and analysis on the first image data to identify the anatomical parts of the subject based on stored characteristics of anatomical parts, generates segmentalized areas of the view of the subject corresponding to image boundaries of the anatomical parts, generates overlay image data of an image containing segment regions corresponding to the segmentalized areas. In particular, the image segmentation unit identifies and differentiates different soft tissues types from one another such as ligaments from muscles from dura and nerve roots. The segmentation unit transmits the overlay image data to the first imaging device as the second image data to effect superimposition of the overlay image on the view of the subject aligned with the segment regions in correspondence with respective ones of the anatomical parts. The segment regions respectively have indicia distinguishing the segment regions apart from each other.

In an embodiment the navigation system further comprises a first display. The image segmentation unit is configured to feed to the first display a first image signal for displaying the view of the subject based on the first image data with the overlay image superimposed on the view of the subject with the segment regions aligned in correspondence with respective ones of the anatomical parts.

In a further embodiment the image segmentation unit is operable to accept user input to alter the overlay image to match alignment of the segment regions of the overlay image with the respective ones of the anatomical parts.

In an embodiment the navigation system further comprises a second imaging device configured to image a second view of the subject and produce second image data corresponding to the second view. The view of the subject via the first imaging device is a first view having a first field of view and a first image plane, and the second view has a second image plane and a second field of view intersecting the first field of view. The second image plane is angled with respect to the first image plane such that a depth of an instrument inserted into the subject along a direction extending into the first image plane is visible. A navigation unit is configured to receive the second image data and transmit a combined image signal to the first display. The combined image signal is based on the first image data, the second image data, and the overlay image data such that the first display produces a picture having the first view with the overlay image superimposed on the first view with the segment regions aligned in correspondence with respective ones of the anatomical parts in a first portion of the first display, and the second view in a second portion of the first display.

In another embodiment of the navigation system the second imaging device is alignable to be at a first position whereat the second view of the subject is imaged and a second position whereat a third view of the subject is imaged having a third field of view and a third image plane, and the third field of view is larger than the first field of view and aligned such that an area beyond the first field of view is imaged. The navigation unit is configured to receive the third image data and transmit the combined image signal to the first display wherein the combined image signal is further based on the third image data. The first display produces a picture having the first view with the overlay image superimposed on the first view with the segment regions aligned in correspondence with respective ones of the anatomical parts in the first portion, the second view in the second portion of the picture, and the third view in a third portion of the picture.

In a further embodiment of the navigation system the first imaging device is a stereoscopic device having two oculars for viewing the first view of the subject. The overlay image is provided in a first ocular of the two oculars and is not provided in a second ocular of the two oculars so as not to obscure the view of the subject in the second ocular. In a particular embodiment, the first imaging device is an endoscope feeding images to first and second displays in place of, or in addition to, the first and second oculars with the overlay image presented in the first display and the unobstructed view presented in the second display.

In some embodiments of the navigation system the first image data includes data corresponding to stereoscopic views of the stereoscopic device. The first display has a 3D displaying capability. A 3D visualization unit receives the first image data, and processes and feeds the first image to produce a 3D display on the first display.

In some embodiments of the navigation system the navigation unit implements an object tracking unit configured to store image data from time sequential images from at least one of the first imaging device and the second imaging device, identify an object captured in the stored image data, and display on any of the first display, the second display a course of travel of the object over a time period of the time sequential images.

In some embodiments the navigation system has a second imaging device configured to image a second view of the subject and produce second image data corresponding to the second view. The view of the subject via the first imaging device is a first view having a first field of view and a first image plane, and the second view has a second image plane and a second field of view intersecting the first field of view, and the second image plane is angled with respect to the first image plane such that a depth of an instrument inserted into the subject along a direction extending into the first image plane is visible. A navigation unit is configured to receive the second image data and implement a model conformance unit configured to analyze the second image data to identify a device captured in the second view, calculate device position data of the device, and adapt a nominal anatomical model to an anatomical structure recognized in the second image data to produce an modified anatomical model and modified anatomical model image data representative of the modified anatomical model. The navigation unit is operable to transmit a combined image signal to the first display. The combined image signal is based on the first image data, the modified anatomical model image data, the device position data, and the overlay image data such that the first display produces a picture having the first view, with the overlay image superimposed on the first view with the segment regions aligned in correspondence with respective ones of the anatomical parts, displayed in a first portion of the picture, and an image of the modified anatomical model in a second portion of the picture with a representation of the device superimposed on the image of the modified anatomical model in accordance with the device position data.

In some embodiments of the navigation system the model conformance unit is operable to accept user input to alter the modified anatomical model to match conformance of the modified anatomical model to the anatomical structure recognized in the second image data. In an alternative embodiment the model conformance unit is omitted and the nominal anatomical model is optionally replaced by a reconstructed 3D anatomical model of the actual patient based on data from preoperative CT, MRI, or other types of images.

In some embodiments of the navigation system the image segmentation unit is operable to accept user input to alter the overlay image to match alignment of the segment regions of the overlay image with the respective ones of the anatomical parts.

An embodiment of the present disclosure includes a method for performing a minimally invasive surgery procedure on a subject, comprising providing a first imaging device producing a view of the subject and producing first image data representative of the view, the first imaging device being a stereomicroscope and configured to receive second image data, generate an overlay image from the second image data, and superimpose the overlay image on said view of the subject. Furthermore, the method comprises providing an image segmentation unit configured to receive the first image data, process and analyze e first image data to identify anatomical parts of the subject based on stored characteristics of anatomical parts, generate segmentalized areas of the view of the subject corresponding to image boundaries of the anatomical parts, generate overlay image data of an image containing segment regions corresponding to said segmentalized areas, and transmit said overlay image data to the first imaging device as the second image data to effect superimposition of the overlay image on the view of the subject aligned with the segment regions in correspondence with respective ones of the anatomical parts, The method also comprises generating the segment regions respectively to have indicia distinguishing said segment regions apart from each other, and executing the minimally invasive surgery procedure using a stereomicroscope or endoscope as the first imaging device displaying the overlay image as a guide to identifying anatomical parts. The view of the subject and the overlay image may be displayed via oculars of the stereomicroscope or endoscope and/or on a display or displays embodied as monitors.

Optionally, an embodiment of a method of the present disclosure includes displaying an anatomical model of the subject in conjunction with the view of the subject such that positions of instruments being used on the subject can be identified relative to the anatomical model of the subject. The anatomical model is optionally a nominal model which may or may not be adapted to correspond to the subject. Alternatively, the anatomical model is a 3/2D reconstruction of the actual patient based on data from preoperative CT or MRI images. The anatomical model is used to define a global frame of reference. The first image data of the view of the subject, and optionally the overlay image data are registered relative to the anatomical model.

The system of the present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value, Moreover, all ranges disclosed herein are to be understood to encompass any and all subranges subsumed therein. For example, a range of “1 to 10” includes any and all subranges between (and including) the minimum value of and the maximum value of 10, that is, any and all subranges having a minimum value of equal to or greater than 1 and a maximum value of equal to or less than 10, e.g., 5.5 to 10. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”. Similarly, references to “sagittal” and “posterior” views are intended to relate the relationship of the views, and not a requirement based on a positioning of a subject.

The exemplary embodiments of a surgical system are discussed in terms of medical devices for the treatment of musculoskeletal disorders and more particularly to a spinal surgery system for treating pathologies of the spine and a method for treating a spine. However, the present invention is not limited to such treatment and may be applied in general to surgical procedures other than those directed to treating pathologies of the spine. Further, the present invention may be used in applications other than surgery such as manufacturing equipment or other equipment requiring precise navigation of tools, devices, or other items relative to a frame of reference dictated by a work piece.

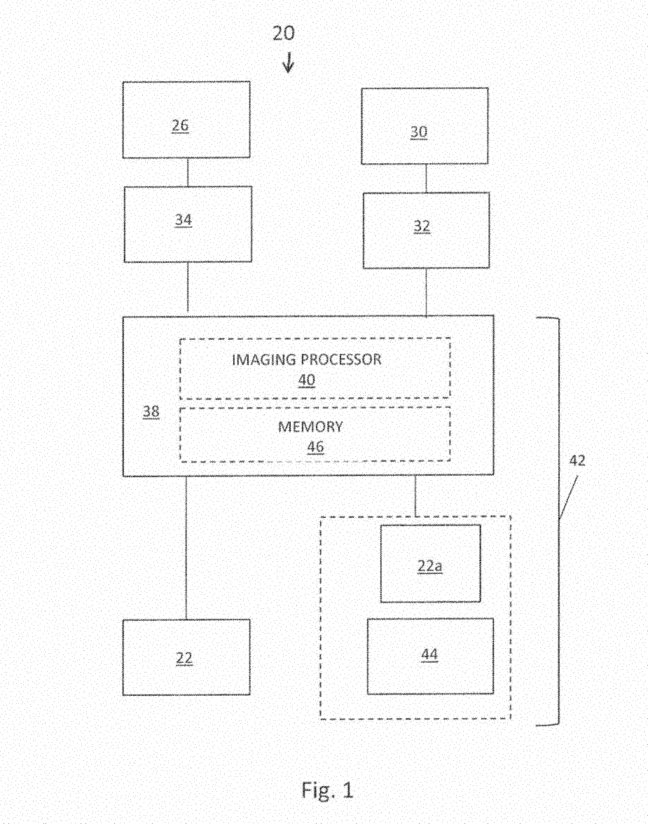

A procedure is performed using a navigation system 20, illustrated in FIG. 1. The procedure is any appropriate procedure, such as a cardiac procedure, ENT, neural procedure, spinal procedure, and orthopedic procedure. The navigation system 20 optionally includes various components, as will be discussed further herein. The navigation system 20 allows a user, such as a surgeon to view on a display 22 a relative position of an instrument to a coordinate system defined by an anatomical structure of a subject upon which the procedure is performed.

It should further be noted that the navigation system 20 is optionally used to navigate or track instruments including: catheters, probes, needles, guide wires, instruments, implants, deep brain stimulators, electrical leads, etc. Moreover, the navigation system 20 is usable on any region of a body of the subject. The navigation system 20 and the various instruments can be used in any appropriate procedure, such as one that is generally minimally invasive, arthroscopic, percutaneous, stereotactic, or an open procedure. Also, instruments discussed herein are only exemplary of any appropriate instrument and may also represent many instruments, such as a series or group of instruments. Identity and other information relating to the instrument can also be provided to the navigation system 20. Further, the information about the instrument can also be displayed on the display 22 for viewing by a surgeon.

Although the navigation system 20 is described herein in conjunction with an exemplary imaging device 26, one skilled in the art will understand that the discussion of the imaging device 26 is merely for clarity of the present discussion and any appropriate imaging system, navigation system, patient specific data, and non-patient specific data is optionally used. Image data, unless explicitly limited herein, is captured or obtained at any appropriate time with any appropriate device.

The navigation system 20 as described herein includes the optional imaging device 26 that is used to acquire pre-, intra-, or post-operative or real-time image data of a patient. The imaging device 26 is, for example, a fluoroscopic x-ray imaging device that may be configured as a C-arm 26 having an x-ray source and an x-ray receiving section. Other imaging devices may be provided such as an ultrasound system, magnetic resonance image systems, computed tomography systems, etc. and reference herein to the C-arm 26 is not intended to limit the type of imaging device. An optional calibration and tracking target and optional radiation sensors can be provided, as understood by one skilled in the art. An example of a fluoroscopic C-arm x-ray device that may be used as the optional imaging device 26 is the “Series 9600 Mobile Digital Imaging System,” from OEC Medical Systems, Inc., of Salt Lake City, Utah. Other exemplary fluoroscopes include hi-plane fluoroscopic systems, ceiling fluoroscopic systems, cath-lab fluoroscopic systems, fixed C-arm fluoroscopic systems, isocentric C-arm fluoroscopic systems, 3D fluoroscopic systems, etc. The imaging device 26 can comprise an O-arm imaging device sold by Medtronic Navigation, Inc. having a place of business in Louisville, Colo., USA. The imaging system 20 can include those disclosed in U.S. Pat. Nos. 7,188,998; 7,108,421; 7,106,825; 7,001,045; and 6,940,941; all of which are incorporated herein by reference.

An optional imaging device controller 34 controls the imaging device 26 to capture the x-ray images received at the receiving section and store the images for later use. The controller 34 is either integrated with or separate from the C-arm 26 and controls positioning of the C-arm 26. For example, as one skilled in the art will appreciate, the C-arm 26 is movable in a direction of an arc or rotatable about a longitudinal axis of a patient, allowing anterior or lateral views of the patient to be imaged. Each of these movements involves rotation about a mechanical axis of the C-arm 26.

The operation of the C-arm 26 is understood by one skilled in the art. Briefly, x-rays are emitted from an x-ray section and received at a receiving section. The receiving section includes an imaging device configured to create the image data from the received x-rays. It will be understood that image data is not limited to that produced by a fluoroscopic device but is optionally created or captured with any appropriate imaging device, such as a magnetic resonance imaging system, a positron emission tomography system, computed tomography, or any appropriate system. It will be further understood that various imaging systems can be calibrated according to various known techniques.

The image data can then be forwarded from the C-arm controller 34 to a navigation unit 38 via a communication system. The navigation system 20 includes an imaging processor 40 and a memory 46. The communication system is optionally any of wireless, wired, a data transfer device, or any appropriate system. A work station 42 includes a first display 22a and a user interface 44 and is optionally integrated with the navigation unit 38. Furthermore, a second display 22 is preferably a large display mounted such that a surgeon or other user of the present invention may readily view the second display 22 while carrying out a surgical or other procedure with the aid of the navigation system 20. It is understood that a single display is an alternative to having the first and second displays 22a, 22. However, having the first and second displays, 22a and 22, is advantageous in that a technician may use the first display 22a in conjunction with the user interface 44 while the surgeon, for instance, views the second display 22. It will also be understood that the image data is not necessarily first retained in the controller 34, but is alternatively directly transmuted to the navigation unit 38.

While the memory 46 is depicted as integral with the navigation unit 38, those skilled in the art will appreciate that memory can also or alternatively be disposed external to the navigation unit 38 as demands or convenience require. For example, data stored in the memory 46 is optionally continuously backed up in a secondary memory so that in the event of a failure of the memory 26 during a procedure, the navigation unit 26 can continue to operate using the memory backup.

The work station 42 provides facilities for displaying the image data as an image on the first and second displays. 22a and 22, saving, digitally manipulating, or printing a hard copy image of the received image data. The user interface 44, which may be a keyboard, mouse, touch pen, touch screen or other suitable device, allows a physician or user to provide inputs to control the imaging device 26, via the C-arm controller 34, or adjust the display settings of the display 22.

While the optional imaging device 26 is shown in FIG. 1, any other alternative 2D, 3D or 4D imaging modality may also be used. For example, any 2D, 3D or 4D imaging device, such as isocentric fluoroscopy, hi-plane fluoroscopy, ultrasound, computed tomography (CT), multi-slice computed tomography (MSCT), T1 weighted magnetic resonance imaging (MRI), T2 weighted MRI, high frequency ultrasound (HIFU), positron emission tomography (PET), optical coherence tomography (OCT), intra-vascular ultrasound (IVUS), ultrasound, intra-operative CT, single photo emission computed tomography (SPECT), or planar gamma scintigraphy (PGS) may also he used to acquire 2D, 3D or 4D pre- or post-operative and/or real-time images or image data of the patient. The images may also be obtained and displayed in two, three or four dimensions. In more advanced forms, four-dimensional surface rendering regions of the body may also be achieved by incorporating patient data or other data from an atlas or anatomical model map or from pre-operative image data captured by MRI, CT, or echocardiography modalities. A more detailed discussion on optical coherence tomography (OCT), is set forth in U.S. Pat. No. 5,740,808, issued Apr. 21, 1998, entitled “Systems And Methods For Guiding Diagnostic Or Therapeutic Devices In Interior Tissue Regions” which is hereby incorporated by reference. Additionally, details of operation of imaging equipment and navigation techniques are disclosed in U.S. Patent Publication US2014/0081128, published Mar. 20, 2014, entitled “Automatic Identification of Instruments Used With A Surgical Navigation System,” U.S. Pat. No. 8,543,189, issued Sep. 24, 2013, entitled “Method And Apparatus For Electromagnetic Navigation Of A Magnetic Stimulation Probe,” U.S. Patent Publication US2012/0194183, published Aug. 2, 2012, entitled “Image Acquisition Optimization,” U.S. Patent Publication US2014/0081354, published Mar. 20, 2014, entitled “Assignment And Manipulation Of Implantable Leads In Different Anatomical regions With Image Background,” U.S. Patent Publication US2012/0250822, published Oct. 4, 2012, entitled “X-ray Imaging System And Method,” and U.S. Patent Publication US2012/0330134, published Dec. 27, 2012, entitled “Interventional Imaging,” each of the foregoing being herein incorporated by reference.

Image datasets from hybrid modalities, such as positron emission tomography (PET) combined with CT, or single photon emission computer tomography (SPECT) combined with CT, can also provide functional image data superimposed onto anatomical data to be used to confidently reach target sites within the patient. It should further be noted that the optional imaging device 26, as shown in FIG. 1, provides a virtual bi-plane image using a single-head C-arm fluoroscope as the optional imaging device 26 by simply rotating the C-arm 26 about at least two planes, which could he orthogonal planes to generate two-dimensional images that can be converted to three-dimensional volumetric images. By acquiring images in more than one plane, an icon representing the location of an impacter, style, reamer driver, taps, drill, deep brain stimulators, electrical leads, needles, implants, probes, or other instrument, introduced and advanced in the patient, may be superimposed in more than one view on the display 22 allowing simulated bi-plane or even multi-plane views, including two and three-dimensional views.

The navigation system 20 further optionally comprises a stereomicroscope 30 equipped to receive images from within a cannula of a laparoscope. The stereomicroscope 30 has a control unit 32 which digitizes images and transmits the corresponding image data to the navigation unit 38. The stereomicroscope 30 further includes image superposition capability which allows the control unit 32 to receive image data and superimpose the corresponding image on the field-of-view (FOV) of either one or both of the oculars of the stereomicroscope as is further discussed below. While a stereoscopic view has advantages over a monoscopic view, as an alternative, a monoscopic microscope may also be employed.

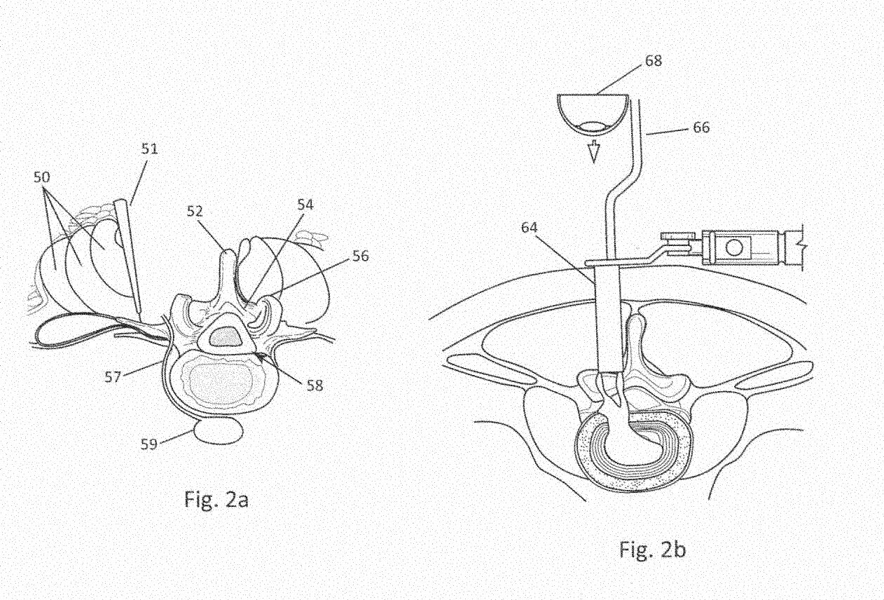

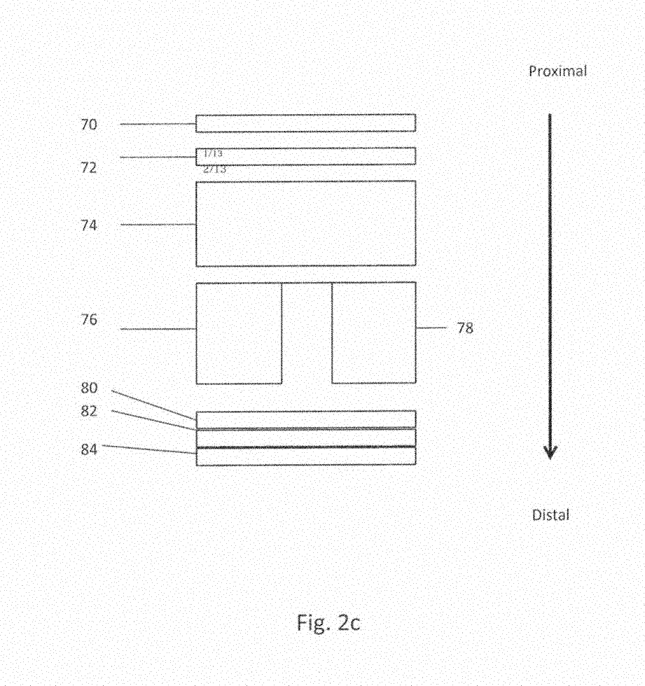

Referring to FIG. 2a, an exemplary open spinal surgical procedure is shown. After an incision is made in overlying skin, muscle 50 is dissected away from a spinous process 52 and lamina 54 by a scalpel 51 thereby exposing the spinous process 52 and lamina 54 so that a bur may be used to effect decompression of spinal stenosis. Further shown for reference are the facet joint 56, segmental spinal artery 57, spinal nerves 58, and aorta 59. Referring to FIG. 2b, minimally invasive surgery (MIS) is shown wherein a small incision is made through which a cannula 64 of a laparoscope is passed to provide access to the spinous process and lamina for a surgical tool 66. In order to illustrate the viewing requirements, an eye 68 is shown above the cannula 64 thus illustrating how narrow an FOV is when the spinous process is viewed through the cannula 64. Now referring to FIG. 2c, a surgical approach through the anatomical structure surrounding and including the spine is schematically illustrated proceeding from a proximal position at a back of a subject to a distal position within the spine. When conducting MIS the cannula is passed through skin 70, fascia 72, muscle 74, muscle 76, and bone 78, to arrive at a layer of ligamentum flavum (LF) 80. Subsequently, the ligamentum flavum 80 is incised to expose epidural fat 82, after which dura 84 is reached. As the surgeon progresses, the navigation system optionally polls the surgeon to accept input identifying landmark structures as may be required to build a model of the surgery. Optionally, the course of the surgery is compared to a nominal or “perfect” surgery.

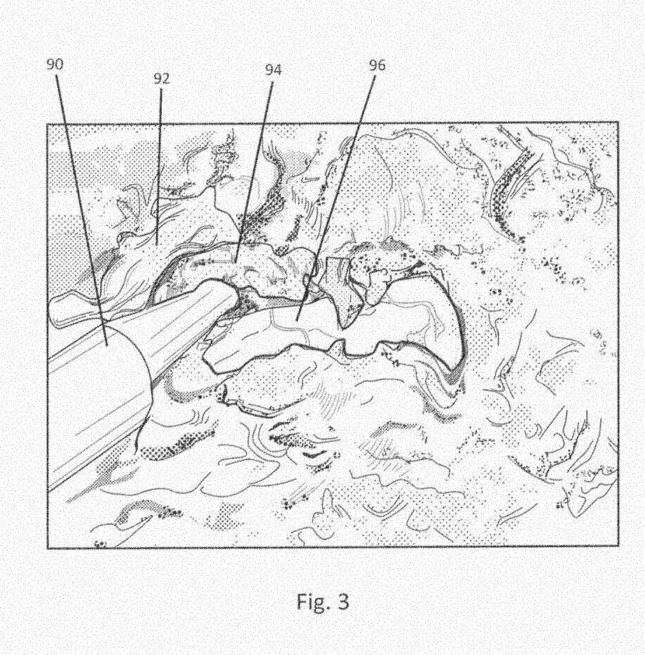

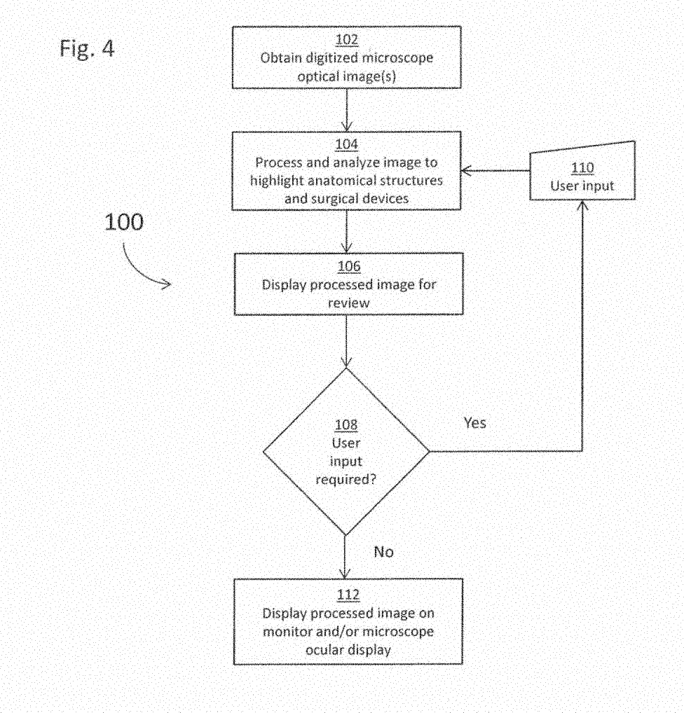

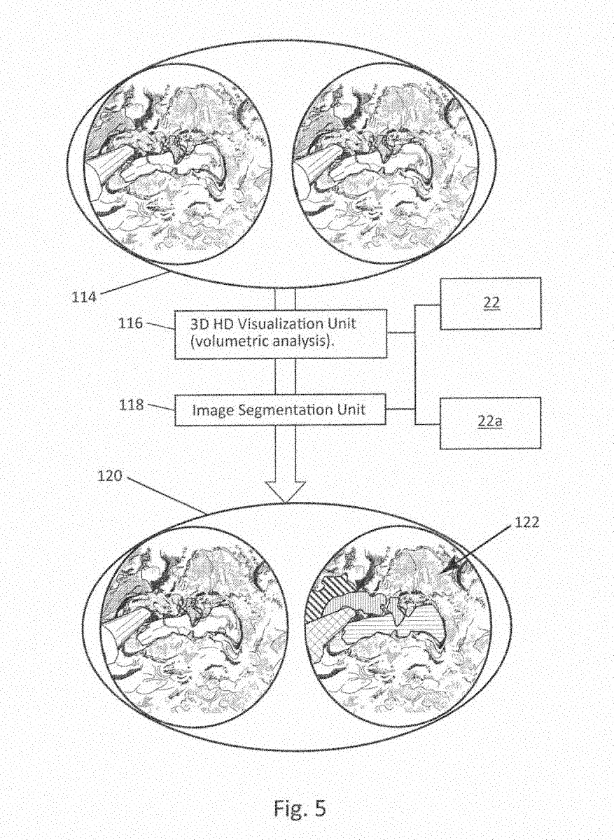

Referring to FIG. 3, a view through the stereomicroscope 30 is shown wherein a bur tool 90, ligamentum flavum 92, epidural fat 94, and the dura 96 are visible. During surgery, it is sometimes difficult to differentiate between these various tissues. Hence, the navigation system 20 of the present disclosure provides guidance by identifying the tissues as related in FIGS. 4-6. In FIG. 4 a microscope image processing procedure 100 is shown which begins in step 102 wherein the navigation unit 38 of FIG. 1 receives digitized microscope optical images from the control unit 32 of the stereomicroscope 30. An example of these images, as seen through the stereomicroscope 30, is shown in a first stereomicroscope view 114 of FIG. 5, The stereomicroscope controller 32, shown in FIG. 1, digitizes the first stereomicroscope view 114 and the resultant digitized image data is passed to a 3D HD visualization unit 116 wherein images are processed to effect 3D display and optionally volumetric analysis. The processed digitized image data is optionally directed to either or both the first and second displays, 22 and 22a, to produce a 3D display of the first stereomicroscope view 114. The 3D HD visualization unit 116 is implemented in the navigation unit 38 by programming stored in the memory 46 operating the imaging processor 40 but may alternatively be implemented as a standalone unit.

The processed image data in step 104 is next directed to an image segmentation unit 118 which effects segmentation of structures in the digitized images and which identifies anatomical structures such as different tissue types, bones or other anatomical parts as colorized segments 122 shown in a second stereomicroscope view 120 of FIG. 5. The segments 122 are represented in overlay data. In step 106 the image segmentation unit 118 combines the processed image data and the overlay data and displays the resultant image for review.

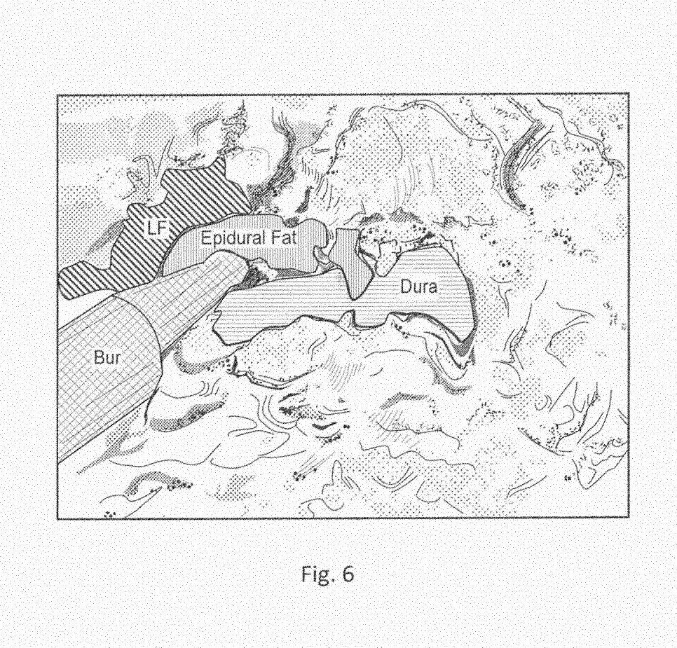

Alternatively to coloring or concurrently therewith, the segments 122 are optionally identified by patterning, labeling, or varying grayscale shading. The image segmentation unit 118 is implemented in the navigation unit 38 by programming stored in the memory 46 operating the imaging processor 40, or is alternatively implemented as a standalone unit. Identification of tissues and anatomical parts is effected by comparison of characteristics of the segmented structure with characteristics of tissues and anatomical parts stored in the memory 46. Additionally, surgical tools, such as a bur, will optionally have characteristics stored in the memory 46 for identification purposes. For further aid to the surgeon, a view is optionally displayed on the second display 22 wherein the colorized segments are labeled as “II”, “Epidural Fat”, “Dura”, and “Bur” as shown in FIG. 6. Additionally, the view of FIG. 6 is further shown in the first display 22 for viewing by a technician controlling the navigation system 20. Those skilled in the art will appreciate that various methods exist for performing the 3D HD visualization processing, volumetric analysis, and image segmentation. Details of exemplary approaches are described in U.S. Pat. No. 7,760,941, issued Jul. 20, 2010, entitled “Method And Apparatus Of Segmenting An Object In A Data Set And Of Determination Of The Volume Of Segmented Object,” U.S. Pat. No. 7,576,740, issued Aug. 18, 2009, entitled “Method Of Volume Visualization,” U.S. Pat. No. 8,295,561, issued Oct. 23, 2012, entitled “Marking A Location In A Medical Image,” and U.S. Pat. No. 6,985,612, issued Jan. 10, 2006, entitled “Computer System And Method For Segmentation Of Digital Image,” each of the foregoing patents being herein incorporated by reference. Alternative methods and hardware, other than those described in the foregoing noted patents, are optionally employed in the present invention and are considered to be within the scope and spirit of the present invention.

Returning to FIG. 4, in step 108 a user reviews the resultant image to determine if the segments 122 of the overlay data are properly aligned or if adjustment is necessary. If the determination is positive, the user inputs data to adjust the alignment of the segments 122 in step 110. If the determination is negative, the processed image data and overlay data is fed to be displayed on either or both of the first imaging device 30, e.g., stereomicroscope, or endoscope, and/or the first display 22 in step 112.

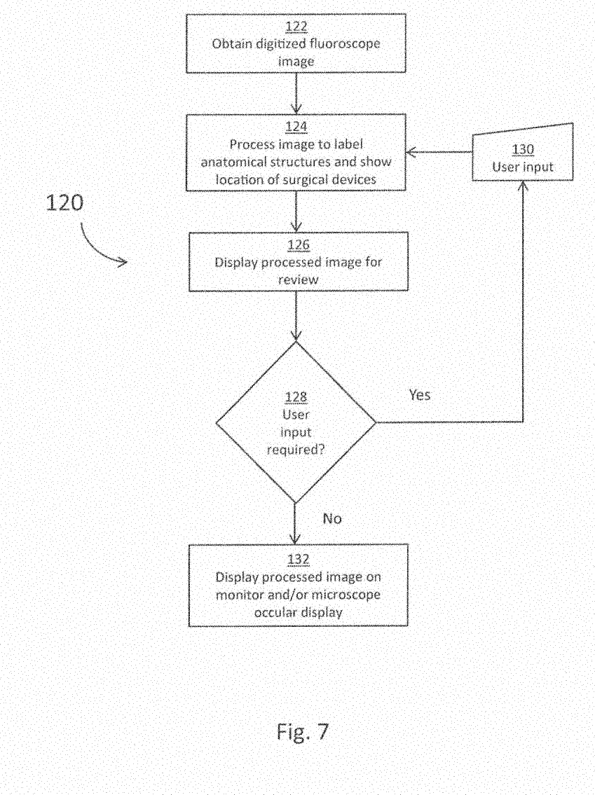

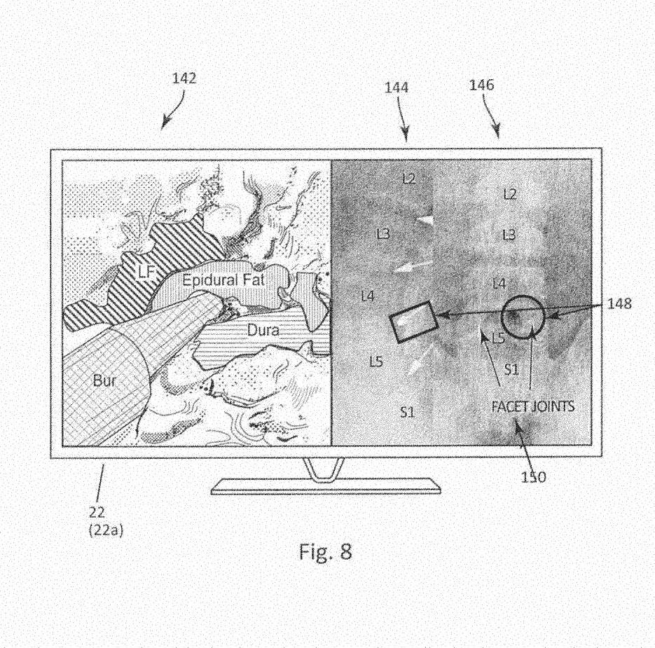

Moving on to FIGS. 7-9, processing of digitized image data from the imaging device 26 of FIG. 1 is described. In the present exemplary embodiment the imaging device 26 is a fluoroscope and is thus referred to herein with the understanding that other types of imaging devices are optionally substituted in place of a fluoroscope. An imaging device data process 120, for processing digitized image data from the fluoroscope, is shown in FIG. 7. The imaging device data process 120 is executed by an image recognition unit 170 which is implemented in the navigation unit 38 by programming stored in the memory 46 operating the imaging processor 40. In step 122, digitized fluoroscope image data is obtained from the imaging device controller 34, if present, or alternatively directing from the imaging device 25. The digitized fluoroscope image data is next processed in step 124 wherein image recognition processing identifies anatomical structures and applies appropriate labeling. The identification is based on characteristics of the anatomical structures stored in the memory 46 to which anatomical structures in the digitized image are compared. The result is shown in FIG. 8, wherein a right side of the display 22 (22a) shows a sagittal view fluoroscope image 144 and a posterior view fluoroscope image 146 with labeling applied to the vertebra L2-L5 and S1. Additionally, further structural features are optionally identified such as facet joints 150 for example. Still further, the image recognition processing identifies the position of the cannula 64, shown in FIG. 2b, as graphic symbols 148 on the images. The cannula 64 is optionally made of a substantially radio transparent plastic having metal registration markers embedded therein which the image recognition processing recognizes and replaces with the graphic symbols 148 in the views. Alternatively, the cannula 64 is formed of a metal, or other radio opaque material, and the view includes the image of the actual cannula 64 rather than a substituted graphic symbol. Those skilled in the art will appreciate, in light of the present disclosure, that various known image recognition methods may be employed wherein imaged structures are compared to models stored in a database to effect recognition. As an example, a method is disclosed in U.S. Patent Publication US2013/0287276, published Oct. 31, 2013, entitled “Image Creation, Analysis, Presentation, And Localization,” which is herein incorporated by reference. However, the present disclosure is not limited to methods described therein and numerous alternative methods are optionally employed.

The sagittal view fluoroscope image 144 and the posterior view fluoroscope image 146 have image planes which are respectively ideally at 90° and 0° with respect to an image plane of the stereomicroscope 30. However, it will be appreciated by those skilled in the art in view of this disclosure that the image planes need not be exactly orthogonal or parallel. Hence, the present disclosure includes angles of the fluoroscope image plane with respect to the stereomicroscope image plane that are in the range of 0° to 90°. For example, the sagittal view fluoroscope image 144 is considered to be in a first range of 90° to 45° with respect to the image plane of the stereomicroscope while the posterior fluoroscope view 146 is considered to be in a second range of 45° to 0° with respect to the image plane of the stereomicroscope. More preferably, the first range is 90° to 55°, and still more preferably in the range 90° to 65°, and yet further preferred is 90° to 75°. Likewise, the second range is 35° to 0°, and still more preferably in the range 25° to 0°, and yet further preferred is 15° to 0°.

As further shown in FIG. 7, in step 126 the image recognition result is displayed for review by a system operator, such as a doctor or technician. The result is displayed on the first display 22a for a technician to verify accuracy of the result. In step 128 a determination is made by the system operator as to whether the result requires modification. If the determination is positive, flow proceeds to step 130 wherein the system operator provides guidance input for the image recognition processing via the user interface 44. Flow then returns to step 124 for further processing. If the determination in step 128 is negative, the results are displayed on the second display 22 for viewing by the surgeon.

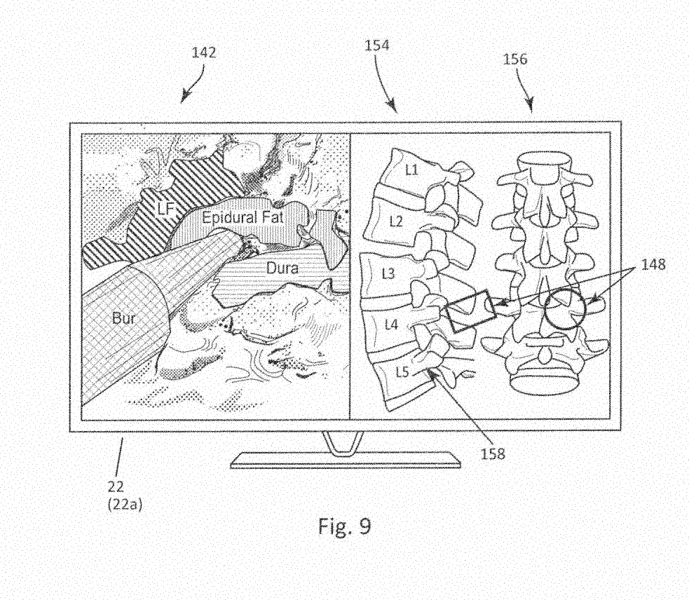

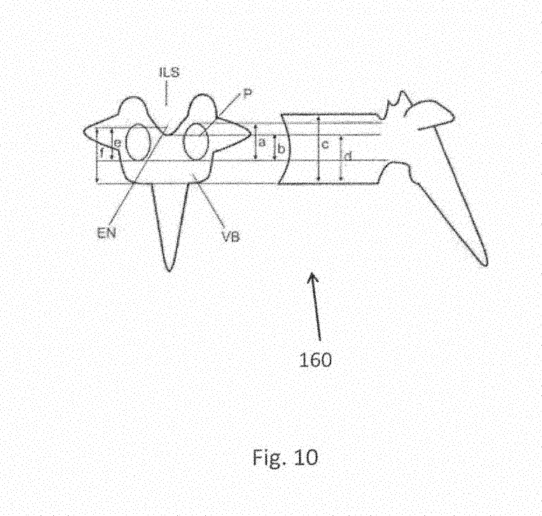

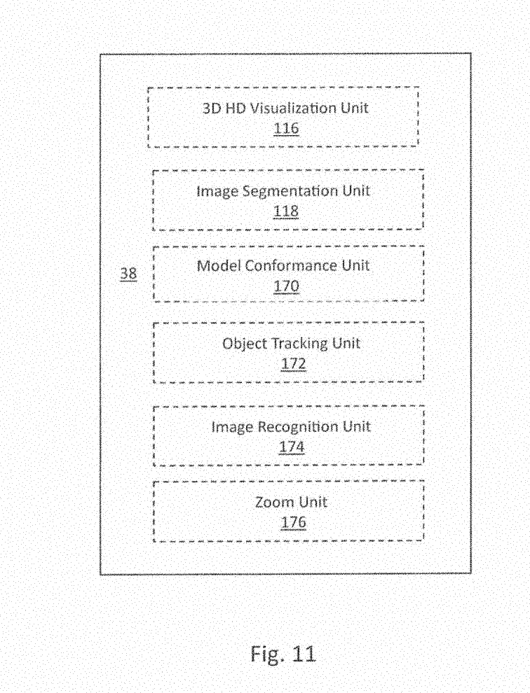

Referring to FIGS. 9 and 10, an alternative presentation method is shown wherein a nominal spine model is used as a reference for indicating position of the cannula 64 or other device. Such a display may provide greater clarity than an actual fluoroscope or other acquired image of the spine. Since the spine model is nominal, in order to make it correspond to the actual subject, the system operator provides user input to modify the spine model based on the imaging provided by the imaging device 26, In FIG. 10, a nominal vertebra model 160 is shown with various dimension variables. The navigation unit 38 implements a model conformance unit 170, shown in FIG. 11, by programming stored in the memory 46 operating the imaging processor 40. The model conformance unit 170 accepts system operator or doctor inputs of actual values based on the image from the imaging device 26 to conform the model to the actual subject. The input is accepted by a model conformance process executed by the model conformance unit 170. The system operator or doctor optionally provides input to identify levels. The model conformance process optionally reads in fluoroscope image data and identifies common landmarks such as, for example and without limitation, pedicles, endplate location, and anterior wall location and modifies positioning of corresponding landmarks on the vertebra model 160. Thus, the model conformance process is used to upsize or downsize the nominal spine model to match the patient, and enable even better anatomical understanding. The modified model is then used in the first and/or second display 22 (22a). As shown in FIG. 9, a sagittal view 154 and a posterior view 156 are presented. Alternatively, if the nominal model is acceptable, user input conforming the vertebra model 160 may be omitted. As a still further alternative a reconstructed 3D anatomical model of the actual patient based on data from preoperative CT or MRI images is used in place of a nominal model or adapted nominal model.

A further feature of the present disclosure includes instrument tracking. As a surgeon executes a procedure, frames of image data from the imaging device 26 is stored in the memory 46. The imaging device 26 periodically takes images which are processed as discussed above and stored in the memory 46 so that a time sequenced record of the procedure is produced. An object tracking unit 172, shown in FIG. 11, is implemented in the navigation unit 38 via programming stored in the memory 46 operating the imaging processor 40. The object tracking unit 172 correlates positions of objects from one point in time to the next. For example, and without limitation, the object tracking unit 172 produces a path history of the bur 90, shown in FIG. 3, throughout a procedure is optionally displayed so that a surgeon can verify that all planned burring has been completed. Likewise, an image of a vertebra operated upon to effect decompression is optionally displayed wherein areas of the vertebra removed are sequentially illustrated. This type of display is sometimes referred to as a “Bone Eraser.” While the present example utilizes repeated imaging by the imaging device 26, the present invention further optionally includes a tracking system using a localizer to track position of an instrument. This system and method of instrument tracking is further detailed in U.S. Patent Publication US2014/0081128, published Mar. 20, 2014, entitled “Automatic Identification of Instruments Used With A Surgical Navigation System,” which is herein incorporated by reference.

Referring to FIG. 11, an alternative diagram of the navigation unit 38 is presented. The navigation unit 38 is shown including the 3D HD visualization unit 116, the image segmentation unit 118, the image recognition unit 174, the object tracking unit 172, the model conformance unit 170, and a zoom unit 176. As related above, the navigation unit 38 of the present disclosure, via operation of the imaging processor 40 in conjunction with model data, anatomical characteristic data, and programming stored in the memory 46, implements the 3D HD visualization unit 116, the image segmentation unit 118, the model conformance unit 170, the image recognition unit 174, the object tracking unit 172, and the zoom unit 176, It is understood that various ones of these units may be optionally implemented physically external to the navigation unit 38 and interconnected to the navigation unit. Hence, for purposes of interpreting the appended claims, a navigation unit “having” or “including” any of the aforesaid functional units is considered to include a navigation unit interconnected with external versions of the aforesaid units.

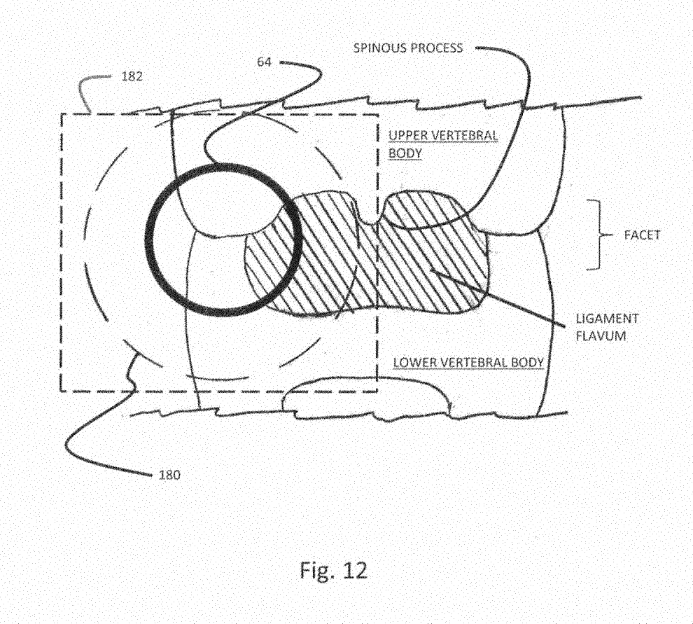

In an embodiment of the present disclosure the navigation unit 38 optionally implements the zoom unit 176, shown in FIG. 11, using programming stored in the memory 46 operating the imaging processor 40. Referring to FIG. 12, operation of the zoom unit 176 is illustrated. The zoom unit 176 provides a zoom feature that magnifies or reduces in size images displayed. The zoom feature permits a view presented in the stereomicroscope or endoscope 30, i.e. the first imaging device, to be zoomed out so that a view provided by the second imaging device 26, for example a fluoroscope, in substantial alignment with the view of the stereomicroscope 30, yet having a larger field of view, supplements the view in the stereomicroscope 30 beyond the restriction of the cannula 64.

FIG. 12 illustrates operation of the zoom unit 176 with reference to a spine structure. The first imaging device 30 provides a view restricted by the cannula 64. Hence, an area inside the cannula 64 in this example is imaged by the stereomicroscope 30 and this is the view available through the stereomicroscope without aid of the zoom unit 176. In order to provide a surgeon with landmark features outside the area of the cannula 64, a size of the image of the first view is reduced so that it occupies an inner portion of the view presented by the stereomicroscope 30. This is shown by a first display view area 180 which represents the view presented by the stereomicroscope 30 when the operation of the zoom unit 176 is implemented. The image from an interior of the cannula 64 is reduced to less than the display view area 180 of the stereomicroscope 30 and an area outside the cannula 64 is filled with the view provided by the second imaging device 26, for example a fluoroscope. The images provided by the stereomicroscope 30 and the second imaging device 26 are adjusted to the same scale, aligned, and combined by the zoom unit 176 to provide a supplemented zoom view within the display view area 180 as shown. Thus, an area greater than the actual FOV of the stereomicroscope 30 can be viewed in a display area of the stereomicroscope 30. Alternatively or in addition to the supplemented zoom view being presented in the display view area 180 of the first imaging device, the stereomicroscope in this example, the zoom unit 176 can present the supplemented zoom view in a second display area 182 of any or all of the first and second displays 22, 22a.

Having described preferred embodiments of the invention with reference to the accompanying drawings, it is to be understood that the invention is not limited to those precise embodiments, and that various changes and modifications may be effected therein by one skilled in the art without departing from the scope or spirit of the invention as defined in this disclosure and the appended claims. Such modifications include substitution of components for components specifically identified herein, wherein the substitute components provide functional results which permit the overall functional operation of the present invention to be maintained. Such substitutions are intended to encompass presently known components and components yet to be developed which are accepted as replacements for components identified herein and which produce results compatible with operation of the present invention.

In summary, it will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

I/We claim:

1. A navigation system for displaying a device relative to a subject and identifying anatomical parts of the subject during a surgical procedure executed by a user upon the subject, comprising:
a first imaging device configured to:
provide a first view of the subject during the surgical procedure, the first view having a first field of view and a first image plane, and
produce first image data representative of the first view during the surgical procedure;
a second imaging device configured to:
provide a second view of the subject during the surgical procedure, the second view having a second field of view and a second image plane, the second image plane being angled relative to the first image plane such that a depth of an instrument inserted into the subject along a direction extending into the first image plane is visible, and
produce second image data representative of the second view during the surgical procedure; and
implement of a navigation unit configured to:
receive the first image data,
implement an image segmentation unit configured to:
process and analyze the first image data to identify the anatomical parts of the subject based on stored characteristics of anatomical parts,
generate segmentalized areas of the first view of the subject corresponding to image boundaries of the anatomical parts,
generate overlay image data of the first image data containing segment regions corresponding to the segmentalized areas,
generate an overlay image from the overlay image data that is directly related to the first view of the subject, and
combine the first image data and the overlay image to effect superimposition of the overlay image on the first view of the subject via alignment of the segment regions to respective ones of the anatomical parts, and
transmit the combined first image data and the overlay image to a first display for display thereon, receive the second image data,
implement a model conformance unit configured to:
process and analyze the second image data to identify a device captured in the second view,
calculate device position data in accordance with the position of the device in the subject, and
adapt a nominal anatomical model to an anatomical structure recognized in the second image data to produce a modified anatomical model and modified anatomical model image data representative of the modified anatomical model, and
combine the modified anatomical image data and the device position data to effect superimposition of the device position data on the modified anatomical model image data, and
transmit the combined modified anatomical image data and
the device position data to the first display for display thereon; and the first display configured to:
display a first picture of the superimposition of the overlay image on the first view, and
display a second picture of the superimposition of the device position data on the modified anatomical model image data.

2. The navigation system of claim 1, wherein the image segmentation unit is operable to accept user input to alter the overlay image to match alignment of the segment regions of the overlay image with the respective ones of the anatomical parts.

3. The navigation system of claim 1, wherein the first image plane and the second image plane subtend an angle which is in a range of 90° to 45°.

4. The navigation system of claim 1, wherein:
the second imaging device is alignable to be at a first position whereat the second view of the subject is imaged and a second position whereat a third view of the subject is imaged having a third field of view and a third image plane, and the third field of view is larger than the first field of view and aligned such that an area beyond the first field of view is imaged, and the navigation unit is configured to receive the third image data, and transmit the third image data for display in a third picture.

5. The navigation system of claim 4, wherein:
the first image plane and the third image plane subtend an angle which is in a range of 0° to 45°; and
the first image plane and the second image plane subtend an angle which is in a range of 90° to 45°.

6. The navigation system of claim 1, wherein:
the first imaging device is a stereomicroscope viewing the subject through a cannula restricting the first field of view; and
the second imaging device is a fluoroscope.

7. The navigation system of claim 6, further comprising:
the first image data including data corresponding to stereoscopic views of the stereoscopic device;
the first display having 3D displaying capability; and
a 3D visualization unit configured to receive the first image data, and process and feed the first image data to produce a 3D display on the first display.

8. The navigation system of claim 7, further comprising the navigation unit implementing an object tracking unit configured to store image data from time sequential images from at least one of the first imaging device and the second imaging device, identify an object captured in the stored image data, and display on the first display a course of travel of the object during a time period of the time sequential images.

9. The navigation system of claim 1, wherein the model conformance unit is operable to accept user input to alter the modified anatomical model to match conformance of the modified anatomical model to the anatomical structure recognized in the second image data.

10. The navigation system of claim 9, wherein the image segmentation unit is operable to accept user input to alter the overlay image to match alignment of the segment regions of the overlay image with the respective ones of the anatomical parts.

11. The navigation system of claim 10, wherein:
the first imaging device is a stereoscopic device and the first image data includes data corresponding to stereoscopic views of the stereoscopic device;
the first display has 3D displaying capability; and
the navigation unit implements a 3D visualization unit configured to receive the first image data, and process and feed the first image to produce a 3D display on the first display.

12. A navigation system for displaying a device relative to a subject and identifying anatomical parts of the subject during a surgical procedure executed by a user upon the subject, comprising:
a first imaging device configured to:
provide a first view of the subject during the surgical procedure, the first view having a first field of view and a first image plane, and
produce first image data representative of the first view during the surgical procedure;
a second imaging device configured to:
provide a second view of the subject during the surgical procedure, the second view having a second field of view and a second image plane, the second image plane being angled relative to the first image plane such that a depth of an instrument inserted into the subject along a direction extending into the first image plane is visible, and
produce second image data representative of the second view during the surgical procedure;
an image segmentation unit configured to:
receive the first image data,
process and analyze the first image data to identify the anatomical parts of the subject based on stored characteristics of anatomical parts,
generate segmentalized areas of the first view of the subject corresponding to image boundaries of the anatomical parts,
generate overlay image data of the first image data containing segment regions corresponding to the segmentalized areas,
generate an overlay image from the overlay image data that is directly related to the first view of the subject, and
combine the first image data and the overlay image to effect superimposition of the overlay image on the first view of the subject via alignment of the segment regions to respective ones of the anatomical parts,
transmit the combined first image data and the overlay image to a first display for display thereon; and
a navigation unit configured to:
receive the second image data,
implement a model conformance unit configured to:
process and analyze the second image data to identify a device captured in the second view,
calculate device position data in accordance with the position of the device in the subject, and
adapt a nominal anatomical model to an anatomical structure recognized in the second image data to produce a modified anatomical model and modified anatomical model image data representative of the modified anatomical model, and
combine the modified anatomical image data and the device position data to effect superimposition of the device position data on the modified anatomical model image data, and
transmit the combined modified anatomical image data and the device position data to the first display for display thereon; and
the first display configured to:
display a first picture of the superimposition of the overlay image on the first view, and
display a second picture of the superimposition of the device position data on the modified anatomical model image data.

13. The navigation system of claim 12, wherein the model conformance unit is operable to accept user input to alter the modified anatomical model to match conformance of the modified anatomical model to the anatomical structure recognized in the second image data.

14. The navigation system of claim 13, wherein the image segmentation unit is operable to accept user input to alter the overlay image to match alignment of the segment regions of the overlay image with the respective ones of the anatomical parts.

15. The navigation system of claim 14, wherein:
the first imaging device is a stereoscopic device and the first image data includes data corresponding to stereoscopic views of the stereoscopic device;
the first display has 3D displaying capability; and
the navigation unit implements a 3D visualization unit configured to receive the first image data, and process and feed the first image to produce a 3D display on the first display.

16. The navigation system of claim 12, wherein:
the first imaging device is a stereomicroscope viewing the subject through a cannula restricting the first field of view; and
the second imaging device is a fluoroscope.

17. The navigation system of claim 16, further comprising:
the first image data including data corresponding to stereoscopic views of the stereoscopic device;
the first display having 3D displaying capability; and
a 3D visualization unit configured to receive the first image data, and process and feed the first image data to produce a 3D display on the first display.

18. A navigation system for displaying a device relative to a subject and identifying anatomical parts of the subject during a surgical procedure executed by a user upon the subject, comprising:
a first imaging device configured to:
produce first image data representative of a first view during the surgical procedure;
a second imaging device configured to:
produce second image data representative of a second view during the surgical procedure; and
implement of a navigation unit configured to:
receive the first image data,
implement an image segmentation unit configured to:
identify the anatomical parts of the subject in the first image data based on stored characteristics of anatomical parts,
generate segmentalized areas corresponding to image boundaries of the anatomical parts based on the first image data,
generate an overlay image from the overlay image data that contains segment regions corresponding to the segmentalized areas, and
combine the first image data and the overlay image to effect superimposition of the overlay image on the first view of the subject via alignment of the segment regions to respective ones of the anatomical parts, and
transmit the combined first image data and the overlay image to a first display for display thereon in a first picture; and
receive the second image data, implement a model conformance unit configured to:
identify a device captured in the second image data,
calculate device position data in accordance with the position of the device in the subject, and
adapt a nominal anatomical model to an anatomical structure recognized in the second image data to produce a modified anatomical model and modified anatomical model image data representative of the modified anatomical model, and
combine the modified anatomical image data and the device position data to effect superimposition of the device position data on the modified anatomical model image data, and
transmit the combined modified anatomical image data and the device position data to the first display for display thereon in a second picture.

19. The navigation system of claim 1, wherein the model conformance unit is operable to accept user input to alter the modified anatomical model to match conformance of the modified anatomical model to the anatomical structure recognized in the second image data.

20. The navigation system of claim 19, wherein the image segmentation unit is operable to accept user input to alter the overlay image to match alignment of the segment regions of the overlay image with the respective ones of the anatomical parts.
